(19) United States
(12) Patent Application Publication
(10) Pub. No.: US 2009/0061009 A1
(43) Pub. Date: Mar. 5, 2009

(54) Composition and Method of Treatment of Bacterial Infections

(76) Inventors: Joseph Schwarz, (US); Michael Weisspapir, (US); Hai Yan Gao, (US)

Correspondence Address: ALPHARX INC., 168 KONRAD CRESCENT, SUITE 200, MARKHAM, L3R 9T9, CA

(21) Appl. No.: 11/846,980

(22) Filed: Aug. 29, 2007

(51) Int. Cl. A61K 9/14; A61K 31/70; A61K 31/7048; A61K 31/496; A61K 31/545; A61K 38/02; A61K 38/12; A61K 31/7036; A61K 38/14; A61K 31/5383; A61K 31/704; A61P 31/04

(52) U.S. Cl. 424/501; 424/489; 514/23; 514/29; 514/254.11; 514/200; 514/2; 514/9; 514/37; 514/8; 514/230.2; 514/34

(57) ABSTRACT

The invention is intended for a treatment of severe infections using an injectable drug-delivery system comprising nanoparticles of a biodegradable polymer with incorporated antibacterial drug.

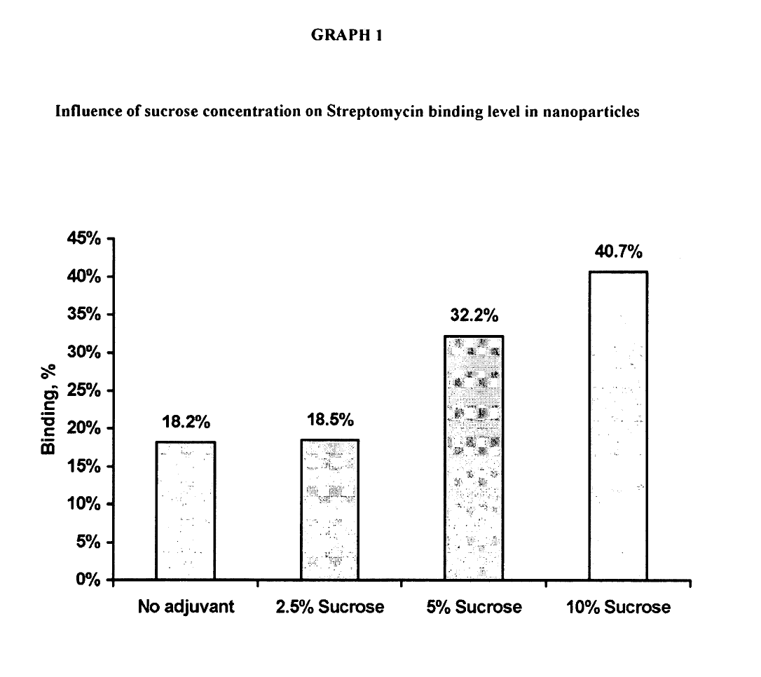

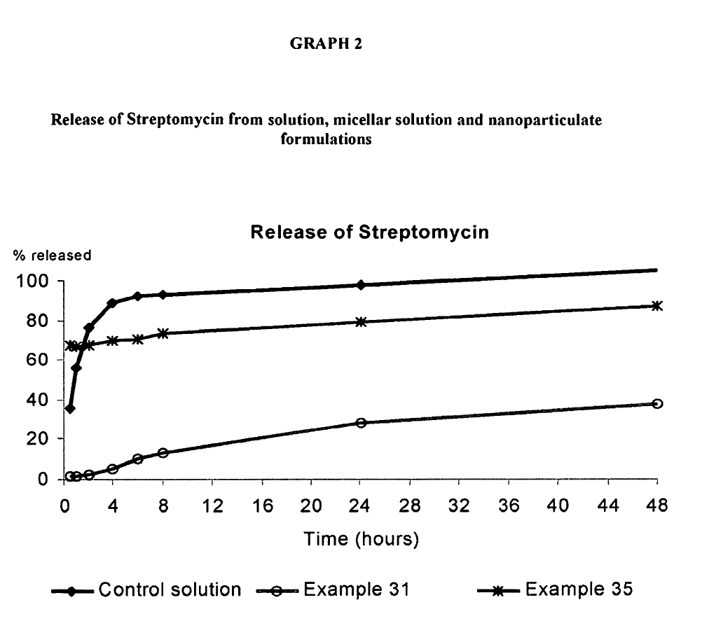

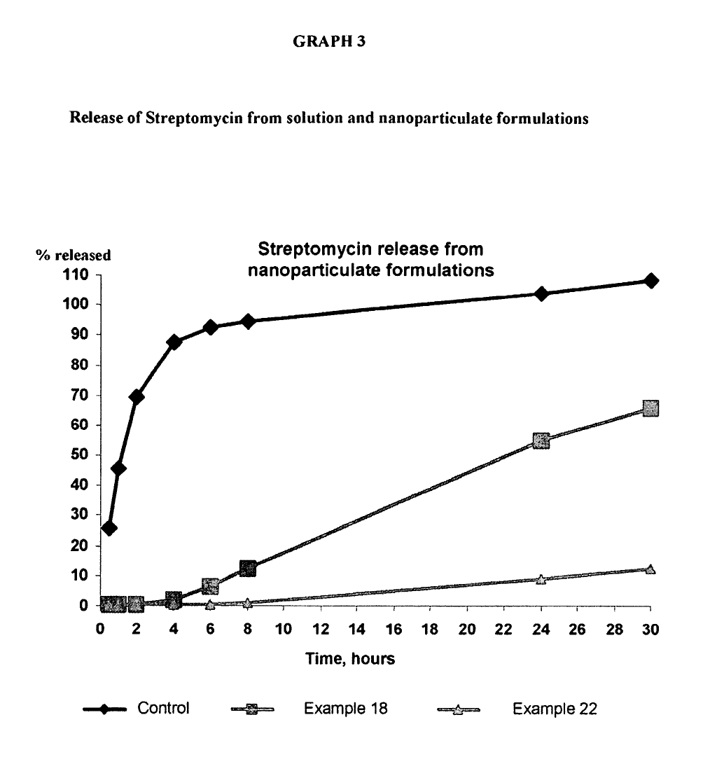

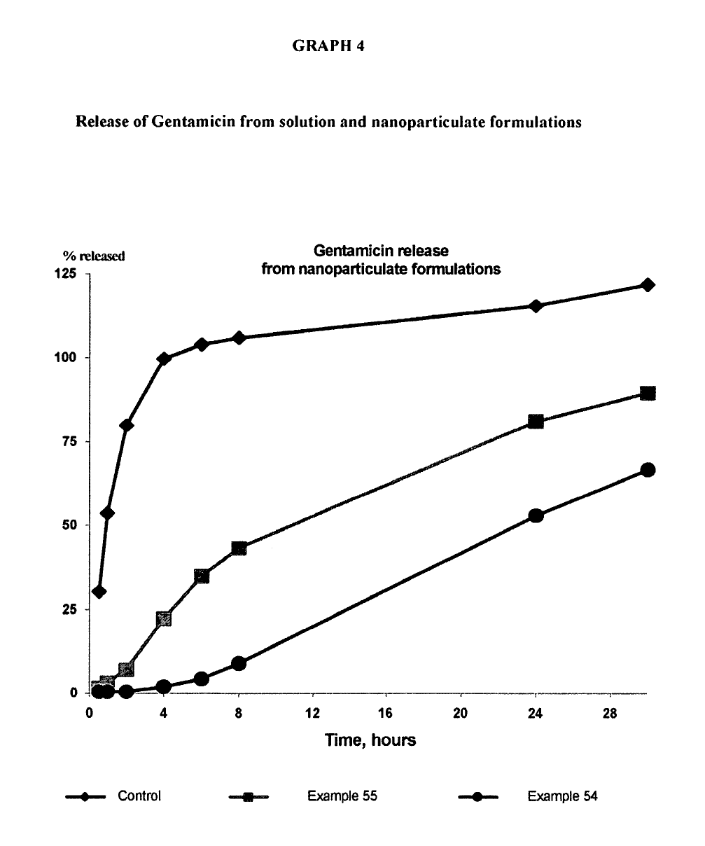

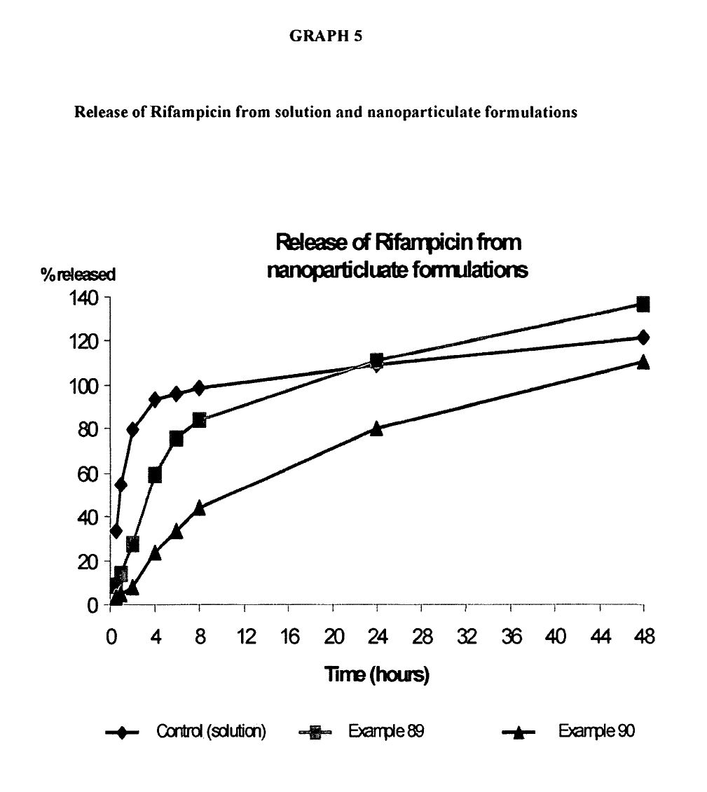

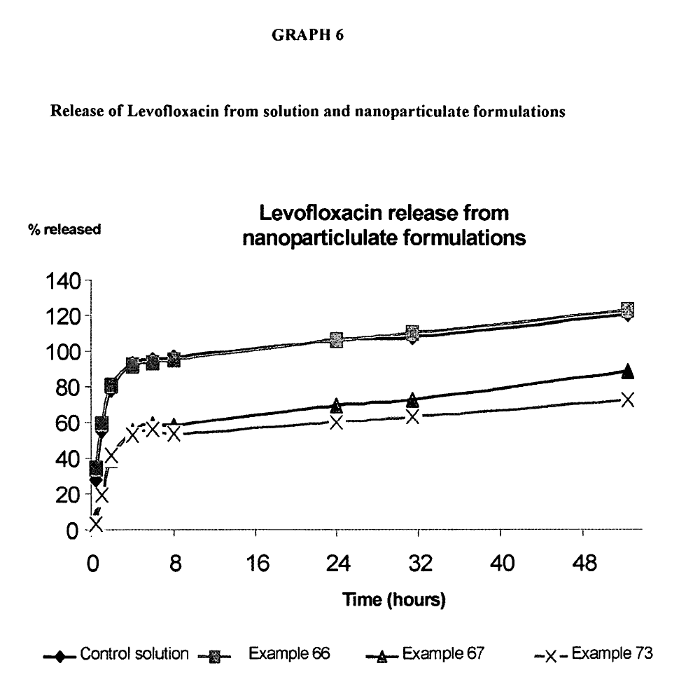

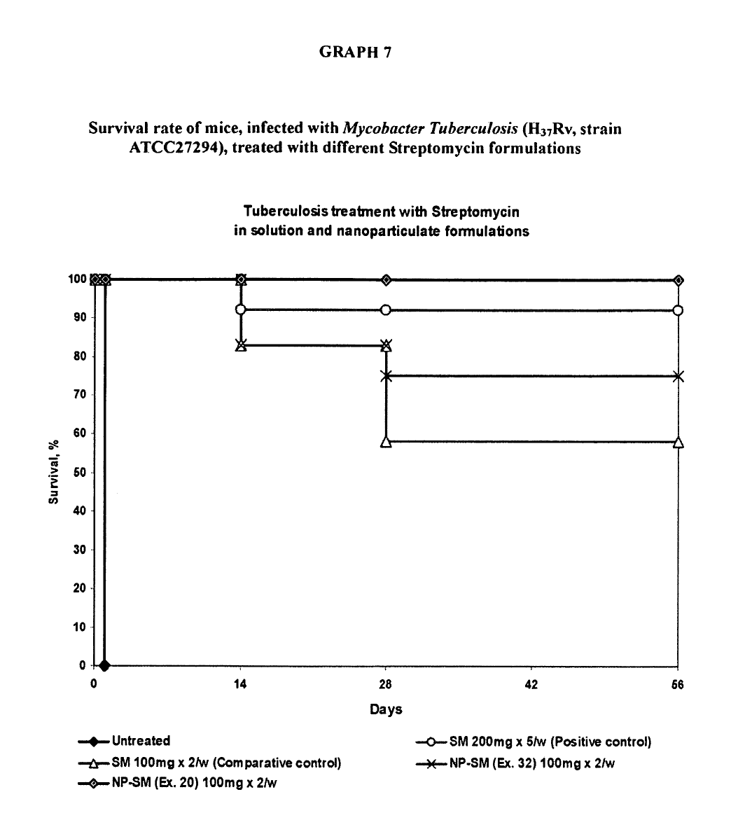

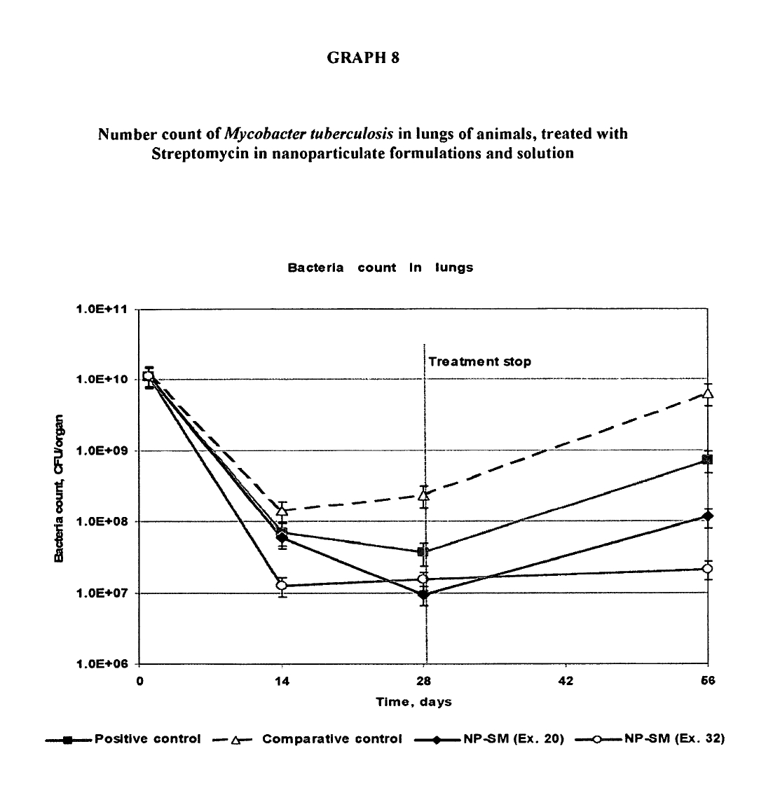

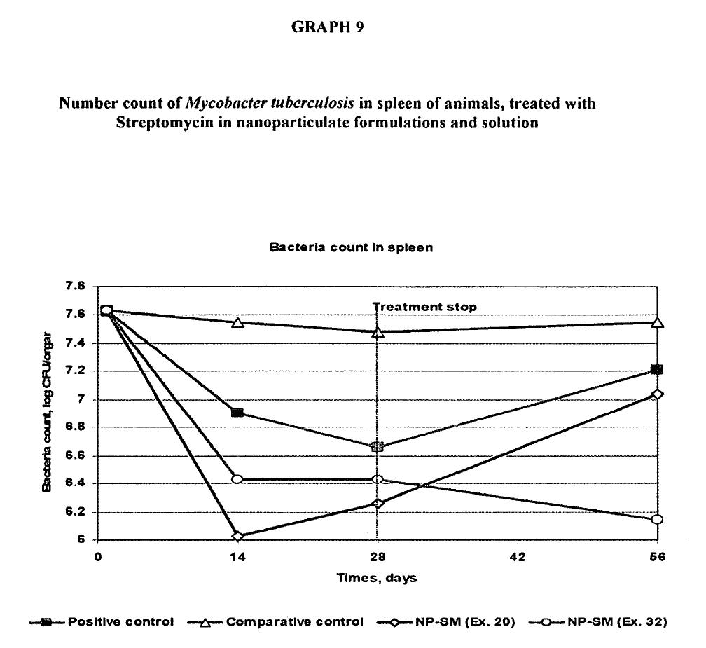

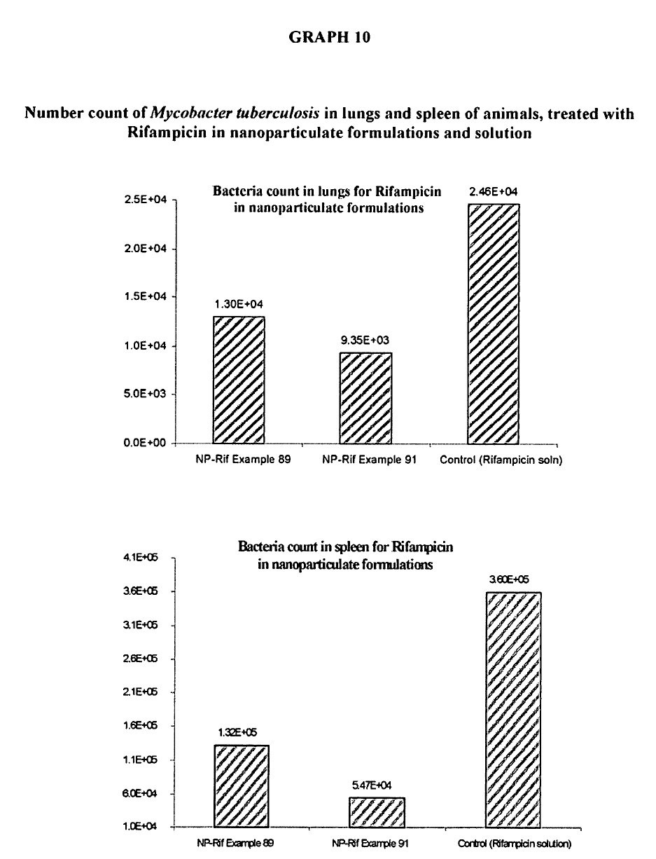

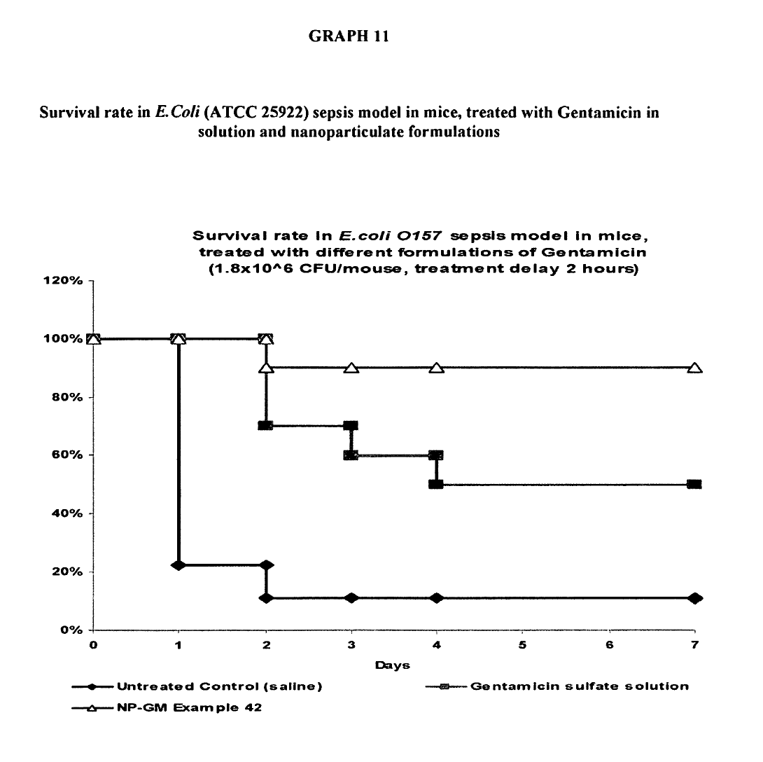

### FIELD OF THE INVENTION

The invention relates to the parenteral delivery of antibiotics incorporated in a biodegradable and biocompatible colloidal composition for the treatment of systemic infections.

### BACKGROUND OF INVENTION

Severe systemic infections, particularly intracellular infections are especially difficult to eradicate because bacteria fight for their survival engage several effective mechanisms against their eradication: inhibition of the phagosome-lysosome fusion, resistance to attack by lysosomal enzymes, oxygenated compounds and defensins of the host macrophages and escape from the phagosome into the cytoplasm. Thus, facultative intracellular bacterial pathogens, such as Salmonella spp., Listeria monocytogenes, Mycobacterium tuberculosis, BrucelIa abortus and obligate intracellular pathogens such as Legionella pneumophila present a major problem. Whilst, intracellular bacteria are found most often in phagocytic cells, they also find their way into non- phagocytic cells such as epithelial cells, hepatocytes and fibroblasts. Facultative intracellular pathogens pose the greatest challenge, as macrophages are not only the cells primarily infected, but also act as a ‘reservoir’ for pathogens which can seed other tissues, leading to a recurrence of infection.

The intracellular activity of antibiotics is dependent on their pharmacokinetic and pharmacodynamic parameters. Poor penetration into cells and decreased intracellular activity are the major reasons for the limited activity of most antibiotics (penicillins, cephalosporins, aminoglycosides) in intracellular infections. An additional difficulty, particularly with classical antibiotic therapy, is that many intracellular bacteria are quiescent or dormant. These bacteria are present in a reversible dormant state and can persist for extended periods without cellular division under a viable but non-culturable state. Also, microorganisms in infected tissues are protected by various biological structures around the infection foci. Indeed, the adhesion properties of bacteria are also expressed by secreting glycocalyx in pathological conditions, providing increased protection and hence increased resistance to antibacterial agents [1]. Despite the discovery of new antibiotics, the treatment of intracellular infections often fails completely to eradicate the pathogens. By loading antibiotics into colloidal carriers, liposomes and nanoparticles, one can expect improved delivery to infected cells [2].

Liposomes loaded with antibiotics have shown higher antibacterial action than antibiotics alone, especially in the case of intracellular infections [3-5]

P. R. J. Gangadharam et al., [6] noted that Streptomycin 100 mg/kg given intramuscularly (IM) five days a week for four weeks caused a significant reduction in the bacterial counts of MAC from spleen, lungs and liver. Alternatively, Streptomycin, given in an encapsulated form in multilamellar liposomes at 15 mg/kg in two intravenous (IV) injections resulted in a greater bacterial count reduction in the same three tissues. The effect of free streptomycin at 150 mg/kg given IM five days a week for eight weeks was compared with 15 mg/kg of streptomycin encapsulated in unilamellar liposomes given IV in four injections (initially and at weekly intervals for three weeks) with no further treatment within the eight week period. Liposome encapsulation resulted in a several-fold increase in the chemotherapeutic efficacy for the liposomal formulation. Similar results were obtained in another study [7] where Mycobacter avium complex infection was treated with liposome encapsulated antibiotics.

Nevertheless, leakage of drug from liposomes during storage limits the potential for the development of a stable and effective liposomal formulation for the delivery of hydrophilic antibiotics. [5-7]

Owing to their polymeric nature, nanoparticles (NP's) may be more stable than liposomes in biological fluids and during storage. Injected nanoparticles, which must be capable of being degraded “in vivo”, allows to avoid side effects resulting from intracellular polymer overload. Polyalkylcyanoacrylate nanoparticles satisfy such requirements; they have been extensively studied because of their ease of manufacture and physicochemical properties [8]. They may be freeze-dried and rehydrated without modifying the particle size and drug content. Their structure allows better retention of the drug within the polymeric network. Subsequently, the nanoparticle network can then be slowly degraded by cellular esterases. Monomers with longer alkyl side chains are preferred, since the acute toxicity of these polymers is greatly reduced [9].

In recent years, biodegradable polymeric NPs have attracted considerable attention as potential drug delivery devices in view of their applications in the controlled release (CR) of drugs, their ability to target particular organs/tissues, as carriers of DNA in gene therapy and in their ability to deliver proteins, peptides and genetic material.

A majority of these NPs are prepared of poly(D,L-lactide), poly(lactic acid) PLA, poly(D,L-glycolide), PLG, poly(lactide-co-glycolide), PLGA, poly(e-caprolactone), PCL or poly(cyanoacrylate) PCA, as well as NPs based on hydrophilic polymers—chitosan, gelatin, sodium alginate and other. The PLA, PLG and PLGA polymers are tissue-compatible and have been used in the past as implantable devices or microparticulate sustained-release formulations in parenteral and implantation drug delivery applications. In addition, poly (e-caprolactone), PCL and poly (alkylcyanoacrylates), PACA, are also being used in preparations of NP's.

Couvreur et al. in [8, 10] described nanoparticles of polyalkylcyanoacrylates loaded with Ampicillin and other antibiotics. These polymers are bioresorbable and have been in use for several years as surgical glues. Ampicillin incorporated into a PIHCA nanoparticle formulation is more than 100 times more effective than free drug in salmonellesis treatment [8-10]. The high efficacy of nanoparticle-bound Ampicillin is observed in the treatment of acute murine experimental salmonellosis and for chronic Listeria monocytogenes infections in mice. This efficacy is attributable to the combined effect of two types of cellular targeting. First, as shown by tissue distribution studies, the binding of Ampicillin to nanoparticles leads to the concentration of drug in the liver and spleen, major foci of infection. Secondly, the cellular uptake of Ampicillin by macrophages is enhanced when the drug is bound to nanoparticles, as compared to uptake in the free form. This involves the uptake of nanoparticles by an endocytotic mechanism, which allows intra-lysosomal localization of the carrier and a subsequent increase in the intracellular concentration of the targeted drug. These results suggest that ampicillin-bound nanoparticles may be effective in the treatment of intracellular bacterial infections in animals and humans.

Colloidal delivery systems,(e.g., nanoparticles), are extensively absorbed within the reticulo-endothelial system of the body, mainly within the mononuclear phagocyte system and thus quickly eliminated from the blood circulation. Such behavior can be modified by the additional coating of nanoparticles with hydrophilic polymers, such as PEG derivatives. Such masking may protect the particles from internalization and increase their circulation time [5, 8]. Some polymers can modify the opsonization process and alter the targeting of such particles, as described in [11] and in U.S. Pat. No. 7,025,991 [12]

In an article of J. Kreuter [13] and U.S. Pat. No. 6,117,454 [14], improved delivery of NP-associated drugs to the brain and CNS using cyanoacrylate nanoparticles is described.

Another article [15] describes polyalkylcyanoacrylate nanoparticles, loaded with the anticancer drug Doxorubicin, which demonstrate improved liver targeting and decreased cardiotoxicity.

In recent years, biodegradable polymeric NP's have attracted considerable attention as potential drug delivery devices in view of their applications in the CR of drugs, their ability to target particular organs or tissues, as carriers of DNA in gene therapy and in their ability to deliver proteins, peptides and genes.

A majority of these NP's are preparations of poly(D,L-lactide) (polylactic acid, PLA), poly(D,L-glycolide), PLG, poly(lacfide-co-glycolide), PLGA, poly(e-caprolactone), PCL and poly(cyanoacrylate), PCA, as well as NPs based on hydrophilic polymers, such as chitosan, gelatin, sodium alginate, albumin among others.

The PLA, PLG and PLGA polymers are tissue-compatible and have a history of prior use as implantable devices or microparticulate sustained-release formulations in parenteral and implantation drug delivery applications. In addition, poly (e-caprolactone), PCL, and poly (alkylcyanoacrylates), PACA, are also being used in preparations of NP's.

Many antibiotics are polar hydrophilic water-soluble compounds and can be easily incorporated into liposomes, (with an internal water phase and an outer bilayer or multiple bilayers of amphiphilic lipids). However, it is often difficult to achieve a high level of drug loading of such water-soluble drugs into polymeric nanoparticles and achieve association level, high enough to obtain the required drug concentration in the target organs, without leakage of incorporated drug from nanoparticles en route.

Penicillins, cephalosporins and aminoglycosides are usually incorporated into NP's with a low loading and binding concentration, due to fast diffusion into the water phase during manufacturing.

Previous attempts at improving the drug loading and binding to NP systems of such water-soluble antibiotics have been largely unsuccessful. Production of nanoparticle preparations, loaded with appropriate therapeutic concentrations of water soluble penicillins, cephalosporins, fluoroquinolones or aminoglycosides, remain a complex task and there are few successful examples. Tracy, M. et al. in U.S. Pat. No. 7,097,857 [16] described a system of PLGA microparticles (>20 mcm size) with biologically active proteins, oligonucleotides and peptides for the targeted delivery. The proteins are stabilized by crosslinking via a complex formation to a stabilizing non-toxic metal cation, selected from the group consisting of Zn⁺, Ca⁺², Cu⁺², Mg ⁺², K⁺ and any combination thereof. Microparticles were prepared in presence of 20 to 60% (by weight of dry microparticle) of water-soluble polymer (PEG-PPO block copolymer, a nonionic surfactant, e.g., Poloxamer) which formed micropores upon hydration. Similar approaches were used in U.S. Pat. Nos. 6,749,866 and 6,500,448 [17,18]

U.S. Pat. No. 5,543,158 [19] describes biodegradable injectable nanoparticles from PLGA-PEG block-copolymer for delivery of antibodies and vaccine adjuvants, containing no additional surfactant.

Proteins and polysaccharides also can be used as constituents of NP matrices. Albumin, chitosan, collagen, alginates and other polymers have been investigated as biocompatible NP components.

Few products containing NP's have received FDA approval for use in humans. ABRAXANE® for Injectable Suspension (paclitaxel protein-bound particles for injectable suspension) is an albumin-bound form of paclitaxel with a mean particle size of approximately 130 nanometers. Transdrug® (Doxorubicin absorbed on Poly (isohexyl)cyanoacrylate nanoparticles) has FDA approval as an orphan drug for liver cancer treatment

Fessi C., et al. (U.S. Pat. No. 5,118,528) developed a method of NP preparation utilizing a precipitation on water dilution process from acetone or other water miscible solvents. This method produces small nanoparticles, but is not suitable for incorporation of water-soluble active compounds.

F. Esmaeili et al. [27] introduced a novel method for the preparation of PLGA nanoparticles loaded with Rifampicin, obtaining a NP compound demonstrating enhanced antibacterial activity. However, concentration of incorporated drug was very low.

US Patent Applications 20030235619 and 20060177495, submitted by Allen C. et al. [21, 22], described PLGA nanoparticles with Taxol, prepared by double emulsification and stabilized with phospholipids and PEG-phospholipids and designed for the incorporation of hydrophobic drugs.

Lipids are also biocompatible and biodegradable and can be used in nanoparticle preparations. Lipid nanoparticles were proposed by Muller in U.S. Pat. No. 6,770,299, as possible delivery vehicles for lipid-drug conjugates [23]. Penkler L, et al. (U.S. Pat. No. 6,551,619) described solid lipid nanoparticles for delivery of Cyclosporin, with improved stability [24]. Gasco R. in U.S. Pat. Nos. 6,685,960 and 6,238,694 described solid lipid nanospheres, suitable for parenteral delivery and fast internalization into cells [25, 26]. Wong H.L. et. al. [28] described preparation of hybrid lipid-polymer nanoparticles, made of polymerized epoxydized unsaturated lipid and stearic acid as lipidic counter-ions, for transport of the anticancer antibiotic Doxorubicin. The authors reached a high drug entrapment concentration and intracellular delivery of the incorporated drug was improved. However, to date, the toxicological properties of the synthesized materials require further evaluation, and preparation of the hybrid polymer is extremely complex.

Vandervoort A. et al. [29] described the interaction of different water-soluble polymeric adjuvants and nanoparticles, stabilized with Polyvinyl alcohol (PVA). In some instances, they observed improved drug stability during lyophilization and reconstitution. However, the drug loading level remained unchanged.

There is high demand for the development of appropriate and safe formulations of antibiotics incorporated in biodegradable nanoparticles, suitable for parenteral administration and effective against intracellular infections. New and effective antibiotic formulations are scarce. Bacterial resistance to existing antibiotics increases by the day. Therefore, the potential to enhance the efficacy of existing antibiotics through the incorporation of biocompatible and biodegradable nanoparticle formulations are of importance to the welfare of all humanity.

### SUMMARY OF THE INVENTION

The invention is intended for the treatment of severe infections using injectable drug-delivery systems comprising nanoparticles of a biodegradable polymer, lipid or combination thereof, with incorporated antibacterial drug. Encapsulation of antibiotics into a biodegradable, nanoparticul ate matrix allows for efficacious treatment of systemic infections caused by pathogenic organisms.

More particularly, the present invention is directed to a treatment of infections, caused by Staphylococcus aureus, Escherichia, Mycobacter tuberculosis, Klebsiella, Streptococci, Salmonella, Listeria, Yersinia, Shigella, Clostridia, Brucella and others, by the administration of a nanoparticulate drug-delivery system incorporating the indicated antibacterial drug. In accordance with an important aspect of the present invention, the drug is water soluble and associated loading of the drug within polymeric nanoparticles is between 10 to 100% of loaded amount. Preferred drugs are antibiotics, selected from classes of aminoglycosides, fluoroquinolones and macrolides.

Another aspect of the present invention is to provide a nanoparticle drug composition, wherein the biodegradable polymer is a polyester-type polymer, such as polylactide, polyglycolide, lactide-glycolide block copolymer, polycaprolactone or poly(gamma-oxybutyrate), or such polymer, combined with a biocompatible lipid matrix.

Yet another aspect of the present invention is to provide a pharmaceutical composition, comprising of biodegradable nanoparticles loaded with an antibacterial drug, which exhibits enhanced antibacterial action in such composition. This composition can be administered to an individual in a therapeutically effective amount to treat an acute or chronic disease or condition and, importantly, the cumulative amount of the drug in nanoparticulate composition, required for treatment, is several times lower than the dose of a conventional formulation.

Another aspect of the present invention is to provide a pharmaceutical preparation comprising biodegradable nanoparticles, containing a water-soluble drug that remains associated with the nanoparticle matrix immediately after administration and is capable of being gradually released in vivo for an extended period of time to treat infection, disease or conditions associated with Staphylococci, Escherichia, Mycobacter tuberculosis, Klebsiella, Streptococci, Salmonella, Listeria, Yersinia, Shigella, Clostridia, Brucella.

Another aspect of the present invention is to provide a biodegradable nanoparticle drug composition, comprising a polyester-type polymer and complex of water soluble antibacterial drug (antibiotic) with a pharmaceutically acceptable counter-ion, such as cholesterol sulfate, tocopherol succinate, cetyl phosphate, aliphatic or aromatic organic acids.

One other aspect of the present invention is to increase the binding capacity of a water soluble antibiotic to a hydrophobic nanoparticle using hydrophilic coadjuvants, which are pharmaceutically acceptable salts, polyols, sugars and polymers, thus providing improved safety, diminished side effects and prolonged sustained release for the composition.

Controlled delivery of antibacterial drug from a biodegradable and biocompatible nanoparticulate delivery system offers profound advantages over conventional antibiotic dosing. Drugs can be used more effectively and efficiently, less drug is required for optimal therapeutic effect and toxicity and side effects can be significantly reduced, or even eliminated, through cellular/tissue targeting. The stability of some drugs can be improved, allowing for a longer shelf-life and drugs with a short half-life can be protected within a nanoparticle matrix from decomposition, enhancing their shelf-life. The benefit of a extended targeted release of drug provides for the maintenance of a continuous therapeutic level of drug, or allows for a pulsatile mode of delivery—each designed, as required, to effect an optimal therapeutic outcome. Inherent in this methodology is a significantly reduced number of drug administrations, perhaps, in some instances, a single dose administration of NP-associated drug, once daily, weekly or for a longer period of time, if appropriate.

Due to low toxicity and high biocompatibility, PLA, PLG, Polycaprolactone and PLGA polymers, these materials were used for preparation of a colloidal delivery system for targeted parenteral antibiotic administration.

Incorporation of antibiotics into nanoparticles having much slower degradation rate, compared with liposomes and polyalkylcyanoacrylates significantly increased the antibacterial activity of their incorporated drugs and provided a substantial decrease in the cumulative effective dose of requisite drug.

Unexpectedly it was found that the addition of some water soluble components to a water-continuous-phase significantly increases drug association with hydrophobic matrices. These water soluble coadjuvants can be physiologically acceptable salts, e.g., sodium phosphate, calcium ascorbate, calcium citrate, gluconate, magnesium sulfate, zinc sulfate, zinc acetate, sodium/potassium citrate and others, or water soluble non-ionic compounds, such as sugars, polyols, di- and polysaccharides, and water soluble oligomers and polymers. Increase of associative binding was not directly associated with ionic strength or “salting-out effect” and was observed in wide pH range, at least from 3.5 to 10. More surprisingly, the use of such water soluble coadjuvants allowed for the stabilization of nanoparticles with antibiotics in a freeze-thawing cycle (normally, a formulation without coadjuvants after 1-2 freezing-thawing cycles demonstrates a tendency to aggregate, increasing the number of particle sizes and with precipitation, while formulations with coadjuvants endure multiple freezing-thawing cycles without changes in physical stability).

NP formulations, prepared according to the invention were tested in animals infected with strongly virulent strains, causing significant clinical symptoms. It was observed that the mortality rate, cumulative antibiotic dose required and frequency of drug administration for NP formulations were significantly lower than for standard treatment procedure for different antibiotics and multiple diseases.

### DETAILED DESCRIPTION OF INVENTION

### Nanoparticles Preparation

Nanoparticles with incorporated antibiotics were prepared by double emulsion technique, or by nanoprecipitation at different drug-to-polymer ratios and water soluble coadjuvants were added to water phase in various concentrations. After elimination of organic solvents, a suspension of formed nanoparticles was concentrated and filtered through a microporous filter membrane. The particle size was measured by photon correlation spectroscopy (Malvern Zetasizer Nano-S). For evaluation of drug loading in NP, a free drug was separated by ultrafiltration (separation membrane with molecular cutoff 30,000 or 300,000 NMWL) and its concentration was measured by HPLC.

Streptomycin in biodegradable polymeric nanoparticles (Examples 1-34). 50-500 mg of antibiotic (Streptomycin sulfate USP) was dissolved in 0.5-1.0 ml of purified water and emulsified in 5-10 ml of organic solvent (water saturated Ethyl Acetate or methylene chloride), containing dissolved D,L-lactide-glycolide copolymer (Resomer® 503H, Boehringer Ingelheim, Germany) with help of short sonication (30 sec) at 20 kHz using titanium indenter or high shear rotor-stator mixer (Ultra-Turrax T10, IKA, Germany). A formed emulsion was added to continuous water phase, containing surfactants and may contain other water soluble adjutants and further homogenized (30 sec. sonication, 3-5 cycles of high pressure homogenization (Avestin Emulsiflex C5 or similar machine). The obtained fine emulsion was evaporated under decreased pressure (2-100 mm) to eliminate organic solvent and concentrate product. The final suspension of nanoparticles was centrifugated (10 minutes, 1000 g) to remove big particles and aggregates, and filtrated through microporous membrane. The particle size was measured by photon correlation spectroscopy (Malvern Zetasizer Nano-S) in water. For the purposes of evaluating a drug as an NP, a free unbond drug was separated by transmembrane ultracentrifugation (separation membrane with molecular cutoff 30,000 or 300,000 NMWL) and its concentration was measured by HPLC.

TABLE 1
Influence of surfactant and adjuvants for binding of Streptomycin to polymeric nanoparticles

| | Example # | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Streptomycin sulfate, mg | 50 | 50 | 50 | 50 | 50 | 50 | 50 | 50 | 50 | 150 | 150 | 150 |
| Polymer | 502S | 502S | 503H | 503H | 503H | 503H | 503H | 503H | 503H | 503H | 503H | 503H |
| Drug:polymer ratio | 1:8 | 1:8 | 1:8 | 1:8 | 1:8 | 1:8 | 1:8 | 1:8 | 1:8 | 1:4 | 1:4 | 1:4 |
| Surfactant(s) | F-68 | F-68 | TPGS | TPGS | TPGS | TPGS | TPGS | TPGS | TPGS | CremEL | CremEL | CremEL |
| | 0.5% | 1.0% | 2% | 2% | 2% | 2% | 2% | 2% | 2% | 2% | 2% | 2% |
| Adjuvant(s) | | | — | Sucrose | Sucrose | MgSO4 | — | — | — | — | ZnSO4 | ZnAc |
| | | | | 2.5% | 10% | 1.0% | | | | | 0.5% | 0.5% |
| | | | | | | | | | | | | Sucrose |
| | | | | | | | | | | | | 10% |
| Stabilizer | | | — | — | — | — | Lipoid | Lipoid | Lipoid | — | | |
| | | | | | | | 75SA | S80H | 75SA | | | |
| | | | | | | | 0.4% | 0.4% | 3.0% | | | |
| Particle size, nm | 86 | 71 | 112 | 217 | 175 | 187 | 103 | 91 | 67, | 215 | 200 | 183 |
| | | | | | | | | | 413 | | | |
| Binding (30K membrane) | 4.3% | 7.2% | 18.2% | 18.5% | 40.7% | 33.9% | 20.0% | 20.9% | 30.9% | 27.5% | 41.2% | 42.9% |

| | Example # | | | | | | | | | | | |
|---|---|---|---|---|---|---|---|---|---|---|---|---|
| | 13 | 14 | 15 | 16 | 17 | 18 | 19 | 20 | 21 | 22 | 23 | 24 |
| Strep- | 150 | 150 | 150 | 150 | 150 | 50 | 50 | 50 | 50 | 50 | 50 | 150 |
| tomycin | | | | | | | | | | | | |
| sulfate, | | | | | | | | | | | | |
| mg | | | | | | | | | | | | |
| Polymer | RG503H | RG503H | RG503H | RG503H | RG503H | RG504H | RG504H | RG504H | RG502S | PCL | RG503H | RG503H |
| | | | | | | | | | | 10K | | |
| Sur- | TPGS | TPGS | TPGS | Tween80 | Tween80 | TPGS | TPGS | BSA | BSA | TPGS | TPGS | CremEL |
| fac- | 1% | 1% | 1% | 2% | 2% | 2% | 1% | 3% | 5% | 1% | 2% | 2% |
| tant(s) | | | | | | | | | | | | |
| Adju- | | Solutol | Solutol | — | ZnSO4 | BSA 3% | BSA 2% | — | | BSA | | ZnSO4 |
| vant(s) | | HS15 | HS15 | | 0.5% | | | | | 3% | | 0.5% |
| | | 1% | 2% | | | | | | | | | Sucrose |
| | | | | | | | | | | | | 10% |
| Sta- | | | — | — | — | — | — | — | | | Na | Na |
| bilizer | | | | | | | | | | | Citrate | Citrate |
| | | | | | | | | | | | 0.4% | 0.4% |
| Particle | 227 | 217 | 212 | 256 | 219 | 176 | 163 | 140 | 227 | 567 | 169 | 241 |
| size, nm | | | | | | | | | | | | |
| Binding | 16.9% | 22.7% | 36% | 5.4% | 16.7% | 33.9% | 42.2% | 59.5% | 79.6% | 86% | 30.9% | 51.4% |
| (30K | | | | | | | | | | | | |
| mem- | | | | | | | | | | | | |
| brane) | | | | | | | | | | | | |

TABLE 2
Influence of counter-ions on association of Streptomycin with nanoparticles

| | Example # | | | | | | | | | | |
|---|---|---|---|---|---|---|---|---|---|---|---|
| | 25 | 26 | 27 | 28 | 29 | 30 | 31 | 32 | 33 | 34 | 35 |
| Streptomycin | 50 | 50 | 50 | 100 | 100 | 100 | 100 | 150 | 50 | 50 | 50 |
| sulfate, mg | | | | | | | | | | | |
| Polymer | RG503H | RG503H | RG503H | RG504H | PCL | RG502S | RG503H | RG502H | RG503H | RG503H | — |
| | | | | | 10K | | | | | | |
| Drug:polymer | 1:4.5 | 1:4.5 | 1:4.5 | 1:8 | 1:4 | 1:8 | 1:8 | 1:4.5 | 1:8 | 1:8 | N/A |
| ratio | | | | | | | | | | | |
| Counter-ion | | 1% | 1% | 2% | 2% | 1% | 2% | 0.5% Na | 0.2% | 0.2% | 2% |
| | | Tocoph. | Tocoph. | NaDOC | NaDOC | NaDOC | NaDOC | Benzoate | KCholSO4 | KCholSO4 | NaDOC |
| | | Succinate | Succinate | | | | | | | | |
| Surfactant(s) | 2% Tw80 | 2% Tw80 | 2% Tw80 | TPGS | TPGS | TPGS | TPGS | CremEL | CremEL | CremEL | TPGS 3% |
| | | | | 3% | 3% | 3% | 3% | 2% | 2% | 2% | |
| Adjuvant(s) | | | Glucose | | | | Sucrose | Sucrose | | Trehalose | Glycerin |
| | | | 5% | | | | 10% | 10% | | 10% | 2.5% |
| Stabilizer | — | — | | | | | | | 0.5% | | 1% Lipoid |
| | | | | | | | | | Lipoid | | S80H |
| | | | | | | | | | S80H | | 0.5% Chol |
| Particle size, | 283 | 256 | 263 | 40.1 | 41.4 | 54.9 | 71.7 | 253 | 181 | 234 | 3.3 |
| nm | | | | | | | | | | | |
| Binding (30K | 16.7% | 27.6% | 32.2% | 76.1% | 89.2% | 68.5% | 96.1% | 19.1% | 42.1% | 55.3% | 70.9% |
| membrane) | | | | | | | | | | | |

Abbreviations:
Polymers: RG502H, RG502S, RG503H, RG504H - copolymers of D, L-lactic and D-glycolic acids (lactide-glycolide copolymers) from Boehringer Ingelheim, Germany.
PCL—Polycaprolactone, MW 10,000 Dalton, Sigma/Aldrich, St Lois, Mo, USA.
TPGS—Tocophersolan USP, PEG-1000 ester of tocopherol succinate (Eastman, UK)
Tween-80—Polysorbate 80 USP;
Solutol HS-15—Ethoxylated (15) 12-hydroxystearic acid, BASF, USA
CremEL—Cremophor EL, Polyethoxylated (35) castor oil USP, BASF;
F-68—Pluronic F68, BASF
Lipoid 75SA, 80H - soy lecithins USP, non-hydrogenated (75% phosphatidylcholine) and hydrogenated (80% phosphatidylcholine), resp., American Lecithin Company
BSA—Bovine Serum Albumin,
NaDOC—Sodium Desoxycholate Tocoph.
Succinate, TocSuc—Tocopheryl acid succinate, Vitamin E succinate, USP
KCholSO4—Cholesteryl sulfate, potassium salt

### EXAMPLES36-55

### Gentamicin in Biodegradable Polymeric Nanoparticles

Nanoparticles with Gentamicin were prepared using the same methods, as for Streptomycin loaded nanoparticles (see examples 1-34). Some of prepared composition are presented in the Table 3.

TABLE 3
Gentamicin in nanoparticulate formulations

| | Example # | | | | | | | | | |
|---|---|---|---|---|---|---|---|---|---|---|
| | 36 | 37 | 38 | 39 | 40 | 41 | 42 | 43 | 44 | 45 |
| Gentamicin | 50 | 50 | 50 | 50 | 50 | 50 | 500 | 500 | 100 | 100 |
| sulfate, mg | | | | | | | | | | |
| Polymer | RG504S | RG504S | RG504S | RG504S | RG504S | RG504S | RG504S | RG503S | RG503S | RG503S |
| Drug:polymer | 1:8 | 1:8 | 1:8 | 1:8 | 1:8 | 1:8 | 1:4 | 1:4 | 1:4 | 1:4 |
| ratio | | | | | | | | | | |
| Counter-ion | | 1% | 1% | 1% | 0.25% | 0.25% | | 0.25% | | 1% |
| | | TocSuc | TocSuc | TocSuc | KCholSO4 | KCholSO4 | | KCholSO4 | | TocSuc |
| Surfactant(s) | 2% F-68 | 2% F-68 | 5% F-68 | 2% F-68 | 0.3% F-68 | 5% F-68 | 1% CremEL | 2% CremEL | 1% | 1% |
| | | | | | | | | | TPGS | TPGS |
| Adjuvant(s) | | | | 0.2% Na | | | Sucrose | Sucrose | 4% | 4% |
| | | | | caprylate | | | 10% | 10% | BSA | BSA |
| Stabilizer | — | — | | | | | 0.1M | 0.1M | | |
| | | | | | | | NaHPO4 | NaHPO4 | | |
| Particle size, nm | 193 | 229 | 167 | 172 | 183 | 155 | 173 | 148 | 245 | 515 |
| Binding | 3.4% | 6.3% | 7.9% | 8.4% | 22.5% | 29.1% | 11.6% | 24.7% | 22.1% | 40.3% |
| (30K membrane) | | | | | | | | | | |

| | Example # | | | | | | | | | |
|---|---|---|---|---|---|---|---|---|---|---|
| | 46 | 47 | 48 | 49 | 50 | 51 | 52 | 53 | 54 | 55 |
| Gentamicin | 50 | 50 | 50 | 50 | 50 | 50 | 100 | 50 | 50 | 50 |
| sulfate, mg | | | | | | | | | | |
| Polymer | RG503S | RG503S | RG503S | RG503S | RG503S | RG503S | RG503S | RG503S | RG502H | PCL |
| | | | | | | | | | | 10K |
| Drug:polymer | 1:8 | 1:8 | 1:8 | 1:8 | 1:8 | 1:8 | 1:4 | 1:8 | 1:8 | 1:8 |
| ratio | | | | | | | | | | |
| Counter-ion | | | | | | | | | 0.25% | |
| | | | | | | | | | KCholSO4 | |
| Surfactant(s) | 1% | 1% | 1% | 1% | 1% | 1% | 1% | 0.5% TP | 2% | 3% BSA |
| | CremEL | CremEL | CremEL | CremEL | CremEL | CremEL | CremEL | GS | Tween80 | |
| Adjuvant(s) | 3% Ca | 2.5% Ca | 3% MgSO4 | 3% ZnSO4 | NaCl | Sucrose | 0.25% | Sucrose | Mannitol | Sucrose |
| | gluconate | ascorbate | | | | 10% | ZnAc | 10% | 5% | 10% |
| Stabilizer | — | 0.25% | | | 0.25% | 0.2% | Sucrose | 0.25% | | |
| | | Lipoid | | | Lipoid | Cholesterol | 10% | Lipoid | | |
| | | S80H | | | S80H | | | S80H | | |
| Particle size, nm | 210 | 261 | 252 | 193 | 274 | 173 | 142 | 228 | 139 | 238 |
| Binding | 43.4% | 27.1% | 63.8% | 77.7% | 27.9% | 35.9% | 23.0% | 47.1% | 50.4% | 69.9 |
| (30K membrane) | | | | | | | | | | |

### EXAMPLES 56-64

### Vancomycin in Biodegradable Nanoparticles

Nanoparticles with Vancomycin were prepared using the same methods, as for Streptomycin loaded nanoparticles (see examples 1-34). Vancomycin dissolved in 0.5-1 ml of water phase or butTer (pH <10), containing surfactant. Some of prepared composition are presented in the Table 4.

TABLE 4
Vancomycin in nanoparticulate formulations

| | Example # | | | | | | | | |
|---|---|---|---|---|---|---|---|---|---|
| | 56 | 57 | 58 | 59 | 60 | 61 | 62 | 62 | 64 |
| Vancomycin | 100 | 100 | 100 | 100 | 100 | 100 | 100 | 100 | 100 |
| HCl, mg | | | | | | | | | |
| Polymer | RG502H | RG502H | RG502H | RG502H | RG502H | RG502H | RG502H | RG502H | RG502H |
| Drug:polymer | 1:4 | 1:4 | 1:4 | 1:4 | 1:4 | 1:4 | 1:4 | 1:4 | 1:4 |
| ratio | | | | | | | | | |
| Counter-ion | | | | 0.5% | 0.5% | 0.25% | 0.5% | 0.5% | 0.5% |
| | | | | TocSuc | TocSuc | TocSuc | KCholSO4 | KCholSO4 | KCholSO4 |
| Surfactant(s) | 2% | 2% | 2% | 2% | 2% | 2% Tween80 | 2% | 2% | 1% TPGS |
| | CremEL | CremEL | CremEL | CremEL | Tween80 | | Tween80 | CremEL | |
| Adjuvant(s) | 0.15M | | 0.05M | 0.05M | Sucrose | Sucrose 10% | Sucrose | Sucrose | Sucrose |
| | NaCl | | Na2HPO4 | Na2HPO4 | 10% | | 10% | 10% | 10% |
| Stabilizer | — | 0.5% Lipoid | 0.5% Lipoid | 0.5% Lipoid | | 0.25% | | | |
| | | S80H | S80H | S80H | | LipoidS80H | | | |
| | | | | | | 0.5% Cholesterol | | | |
| Particle size, | 205 | 146 | 159 | 124 | 137 | 128 | 65.4 | 73 | 79 |
| nm | | | | | | | | | |
| Binding | 0% | 3.1% | 13.5% | 28.1% | 18.7% | 25.1% | 79.3% | 82.1% | 86.8% |
| (300K | | | | | | | | | |
| membrane) | | | | | | | | | |

### EXAMPLES 65-73

### Levofloxacin in Biodegradable Nanoparticles

Nanopailicles with Levofloxacin were prepared using the same methods, as for Streptomycin loaded nanoparticles (see examples 1-34). Levofloxacin was dissolved in water phase with pH adjusted to 2.5 using 1N HCl.

Composition of Example 73 was prepared by precipitation of dissolved combination of polymer, lipid, surfactants, counter-ion and drug from solution in acetone, followed by evaporation of solvent and water.

Some of prepared composition are presented in the Table 5.

TABLE 5
Levofloxacin in nanoparticulate formulations

| | Example # | | | | | | | | |
|---|---|---|---|---|---|---|---|---|---|
| | 65 | 66 | 67 | 68 | 69 | 70 | 71 | 72 | 73 |
| Levofloxacin, mg | 100 | 100 | 100 | 100 | 100 | 100 | 100 | 100 | 50 |
| Polymer | RG504H | RG504H | RG504H | RG504H | RG504H | RG503 | RG504H | RG504H | RG504H |
| Drug:polymer | 1:10 | 1:4 | 1:4 | 1:4 | 1:4 | 1:4 | 1:4 | 1:4 | 1:5 |
| ratio | | | | | | | | | |
| Counter-ion | | 0.2% Benzoic | 0.2% Cetyl | 0.5% | 0.1% | 0.5% | | 0.5% | 0.5% Cetyl |
| | | acid | phosphate | TocSuc | NaDOC | KCholSO4 | | TocSuc | phosphate |
| Surfactant(s) | 3% | 2% Tween80 | 2% Solutol | 0.5% | 2% | 2% TPGS | 1% BSA | 0.5% | 1% Span20 |
| | Tween80 | | HS15 | TPGS | Tween80 | | | TPGS | 1% Tween80 |
| Adjuvant(s) | | | | | Sucrose | 5% PVP | | 1% Solutol | 2.5% Glycerin |
| | | | | | 10% | | | HS15 | |
| Stabilizer | | | | | | | 1% Lipoid | | 1% Lipoid75SA |
| | | | | | | | 75SA | | 1% SuppocireCM |
| Particle size, nm | 199 | 136 | 159 | 152 | 121 | 128 | 248 | 190 | 209 |
| Binding | 3.2% | 7.8% | 27% | 19% | 16.5% | 13.8% | 36.5% | 43.8% | 42% |
| (300K membrane) | | | | | | | | | |

### EXAMPLES 74-81

### Azithromycin in Biodegradable Nanoparticles

Nanoparticles with Azithromycin were prepared using the same methods, as for Streptomycin loaded nanoparticles (see examples 1-34).

Some of prepared composition are presented in the Table 6.

TABLE 6
Azithromycin in nanoparticulate formulations

| | Example # | | | | | | | |
|---|---|---|---|---|---|---|---|---|
| | 74 | 75 | 76 | 77 | 78 | 79 | 80 | 81 |
| Azithromycin, | 100 | 100 | 100 | 100 | 100 | 100 | 100 | 100 |
| mg | | | | | | | | |
| Polymer | RG502H | RG502H | RG502H | RG502H | RG504H | RG503 | RG502H | RG502H |
| Drug:polymer | 1:4 | 1:4 | 1:4 | 1:4 | 1:4 | 1:4 | 1:4 | 1:4 |
| ratio | | | | | | | | |
| Counter-ion | — | 0.5% | 0.5% | 0.5% | | | | |
| | | TocSuc | TocSuc | TocSuc | | | | |
| Surfactant(s) | 2% | 2% | 2% | 2% Tween80 | 1% TPGS | 0.5% TPGS | 2% CremEL | 2% CremEL |
| | Tween80 | Tween80 | Tween80 | | | | | |
| Adjuvant(s) | 10% Sucrose | 10% Sucrose | 10% Sucrose | 10% Sucrose | 1% | 1% BSA | 10% Sucrose | 10% Sucrose |
| | 0.05M | 0.05M | 0.05M | 0.05M | PluronicF68 | | | |
| | NaCitrate | NaCitrate | NaCitrate | NaCitrate | | | | |
| Stabilizer | | | Cholesterol | 1% Lipoid80H | | | 1% Lipoid80H | 0.5% Glyceryl |
| | | | | | | | | distearate |
| Particle size, | 133 | 162 | 148 | 168 | 94 | 112 | 91 | 192 |
| nm | | | | | | | | |
| Binding | 22 | 20.8 | 38.4 | 30.3 | 38.1 | 38.9 | 48.8 | 38.3 |
| (300K | | | | | | | | |
| membrane) | | | | | | | | |

### EXAMPLES 82-88

### Clarithromycin in Biodegradable Nanoparticles

Nanoparticles with Clarithromycin were prepared using the same methods, as for Streptomycin loaded nanoparticles (see examples 1-34).

Formulations of Examples 84 and 85 were prepared by precipitation of dissolved combination of polymer, lipid, surfactants, counter-ion and drug from solution in acetone, followed by evaporation of solvent and water.

Some of prepared composition are presented in the Table 7.

TABLE 7
Clarithromycin in nanoparticulate formulations

| | Example # | | | | | | |
|---|---|---|---|---|---|---|---|
| | 82 | 83 | 84 | 85 | 86 | 87 | 88 |
| Clarithromycin, | 100 | 100 | 50 | 50 | 100 | 100 | 50 |
| mg | | | | | | | |
| Polymer | RG504H | RG504H | PCL 10K | PCL 10K | RG502H | RG503 | RG502H |
| Drug:polymer | 1:4 | 1:4 | 1:8 | 1:4 | 1:4 | 1:4 | 1:4 |
| ratio | | | | | | | |
| Counter-ion | — | 0.5% | | 0.5% | | 0.5% | 0.5% |
| | | TocSuc | | TocSuc | | NaDOC | TocSuc |
| Surfactant(s) | 2% | 2% | 2% Tween80 | 2% Tween80 | 2% CremEL | 0.5% TPGS | 2% CremEL |
| | Tween80 | Tween80 | | | | | |
| Adjuvant(s) | 0.05M | 10% Sucrose | | 1% Pluronic | 10% Sucrose | 1% BSA | 10% Sucrose |
| | NaAcetate | 0.05M | | F68 | | | |
| | | NaAcetate | | | | | |
| Stabilizer | | | 1% Lipoid75SA | Tocopherol | 1% Lipoid75SA | | 1% Precirol |
| | | | | acetate 10% | | | |
| | | | | 1% Lipoid75SA | | | |
| Particle size, | 193 | 222 | 188 | 168 | 113 | 52 | 162 |
| nm | | | | | | | |
| Binding | 17 | 23.5 | 8.4 | 21.7 | 31.8 | 26.9 | 34.3 |
| (300K | | | | | | | |
| membrane) | | | | | | | |

### EXAMPLES 89-96

### Rifampicin in Biodegradable Nanoparticles

Polymeric nanoparticles with Rifampicin and PLGA were prepared using the same methods, as for Streptomycin loaded nanoparticles (see examples 1-34), with methylene chloride as a solvent. Lipid nanoparticles (example 96) were obtained using hot high pressure homogenization.

Some of prepared composition are presented in the Table 8.

TABLE 8
Rifampicin in nanoparticulate formulations

| | Example # | | | | | | | |
|---|---|---|---|---|---|---|---|---|
| | 89 | 90 | 91 | 92 | 93 | 94 | 95 | 96 |
| Rifampicin, mg | 100 | 100 | 100 | 100 | 100 | 100 | 100 | 500 |
| Polymer | RG502H | RG502H | RG502H | RG504H | RG503 | RG502H | RG502H | Synchrowax |
| Drug:polymer ratio | 1:4 | 1:4 | 1:4 | 1:4 | 1:4 | 1:4 | 1:4 | 1:10 |
| Counter-ion | — | 0.5% | 0.5% | | | | | 0.8% TocSuc |
| | | TocSuc | TocSuc | | | | | 0.5% KCholSO4 |
| Surfactant(s) | 2% | 2% | 2% Tween80 | 1% TPGS | 0.5% TPGS | 2% CremEL | 2% | 2% CremEL |
| | Tween80 | Tween80 | | | | | CremEL | |
| Adjuvant(s) | 10% Sucrose | 10% Sucrose | 10% Sucrose | 1% | 1% BSA | 10% Sucrose | 10% Sucrose | 0.5% NaCitrate |
| | 0.05M | 0.05M | 0.05M | PluronicF68 | | | | |
| | NaCitrate | NaCitrate | NaCitrate | | | | | |
| Stabilizer | | Cholesterol | 1% Lipoid80H | | | 1% Lipoid80H | 0.5% | 0.25% Lipoid75SA |
| | | | | | | | Glyceryl | |
| | | | | | | | distearate | |
| Particle size, nm | 133 | 148 | 168 | 94 | 112 | 91 | 192 | 320 |
| Binding | 22 | 38.4 | 30.3 | 38.1 | 38.9 | 48.8 | 38.3 | 93.7 |
| (30K membrane) | | | | | | | | |

### EXAMPLES 97-103

### Doxorubicin in Biodegradable Nanoparticles

Nanoparticles with Doxorubicin were prepared using the same methods, as for Streptomycin loaded nanoparticles (see examples 1-34). Composifions of Examples 100 and 101 were prepared by precipitation of from solution in acetone, followed by evaporation of solvent and water.

Some of prepared composition are presented in the Table 9.

TABLE 9
Doxorubicin in nanoparticulate formulations

| | Example # | | | | | | |
|---|---|---|---|---|---|---|---|
| | 97 | 98 | 99 | 100 | 101 | 102 | 103 |
| Doxorubicin, mg | 20 | 20 | 20 | 20 | 20 | 20 | 20 |
| Polymer | RG502H | RG502H | RG502H | RG502S | RG502H | RG503 | RG502H |
| Drug:polymer | 1:10 | 1:10 | 1:10 | 1:10 | 1:10 | 1:10 | 1:10 |
| ratio | | | | | | | |
| Counter-ion | | | | 0.2% | 0.5% | | 0.5% |
| | | | | Cetylphosphate | TocSuc | | TocSuc |
| Surfactant(s) | 1% | 1% | 1% PluronicF68 | 2% Tween 80 | 2% | 1% HSA | 2% |
| | PluronicF68 | PluronicF68 | | | CremEL | | CremEL |
| Adjuvant(s) | | 1% BSA | 5% Glucose | 0.05M | 10% Sucrose | 0.05M | 10% Sucrose |
| | | | | NaCaprate | | NaCaprate | |
| Stabilizer | | | 0.25% Lipoid75SA | | | | |
| Particle size, nm | 144 | 151 | 174 | 238 | 113 | 186 | 240 |
| Binding | 23 | 29 | 51 | 71.7 | 98.8 | 63 | 99.7 |
| (300K | | | | | | | |
| membrane) | | | | | | | |

Kinetics of release of associated drug from colloidal formulations into phosphate buffered saline (PBS) was investigated using dialysis tube (Spectra pore®) with cellulose membrane (MW cutoff 50,000 Dalton) in USP dissolution apparatus II (paddles, 50 rpm) at 37° C. Results are presented at graphs 2-6.

### Infection Models:

Tuberculosis model: Extremely lethal Mycobacterium tuberculosis strain H₃₇Rv (ATCC 27294) in dose 10⁷ cfu/mice, causing 100% lethality in SPF BALB/C mice in 72 hours after inoculation, was used.

Sepsis (septicemia) model: Escherichia coli O157 was chosen as a model infection being one of the most common nosocomial pathogens. Female BALB/C mice were infected by the intraperitoneal injection of 2.5×10⁸ cells (LD₉₀). Treatment started 2 hours post bacterial inoculation

Pneumonia model: Streptococcus pneumonia serotype 3 strain (ATCC 6303), administrated intratracheally into Swiss Webster mice (10⁵-10⁶ cfu/mice) was used as a model of community acquired pneumonia (CAP), with treatment beginning 24 hours after disease initiation.

Drug-loaded NP formulations and control antibiotics in solution were administrated according to predetermined route and schedule.

Tuberculosis: Streptomycin formulations

SPF BALB/c female mice (18-20 g, n=65) were infected with M. tuberculosis (H₃₇Rv, ATCC27294, 10⁷ CFU/mouse, iv). Poly(lactide-glycolide) nanoparticulate formulations, stabilized with BSA (bovine serum albumin) (Example) and Cremophor (Polyethoxylated castor oil) (Example), were tested. Infected mice (12 per group) were treated IP as follows:
1. Untreated (saline), 5 times per week
2. SM sulfate USP, 200 mg/kg (calc. as streptomycin base), 5 times per week (positive control)
3. SM sulfate USP, 100 mg/kg, twice weekly (comparative control).
4. SM NP Example 20, 100 mg/kg, twice weekly.
5. SM NP Example 32, 100 mg/kg, twice weekly.

SM formulations were administered IP for 28 days. Four mice from each group were assessed for CFU count and organ weights on days 14, 28 and 56.

All animals survived in NP-SM (Example 32) group, received 800 mg cumulative dose of SM, while survival rate for positive control (SM USP, cumulative 4000 mg) was 92%, and for comparative control (SM USP solution, total 800 mg) was 58% only. Bacterial count in lung and spleen was also significantly lower forNP groups.

TABLE 10
Comparative antituberculosis activity of Streptomycin in solution and nanoparticulate
formulations

| | Cumulative dose | Survival | | Bacterial count in lungs, | |
|---|---|---|---|---|---|
| | of SM base, | rate, % | | log CFU (10.06 ± 0.304 at D1) | |
| Groups (n = 12 per group) | mg/kg | D14 | D28 | D28 | D56 |
| Untreated (saline) | 0 | 0 | 0 | NA | NA |
| SM USP solution 200 mg/kg, | 4000 | 92 | 92 | 7.57 ± 0.268 | 8.86 ± 0.18 |
| 5/week (Positive control) | | | | | |
| SM USP solution 100 mg/kg, | 800 | 83 | 58 | 8.37 ± 0.367* | NA |
| 2/week (Comparative control) | | | | | |
| NP-SM (Ex. 20) 100 mg/kg, | 800 | 83 | 75 | 6.97 ± 0.163 | 8.06 ± 0.506* |
| 2/week | | | | | |
| NP-SM (Ex. 32) 100 mg/kg, | 800 | 100 | 100 | 7.18 ± 0.252 | 7.33 ± 0.242* |
| 2/week | | | | | |

*P < 0.05 vs Positive control.

Tuberculosis: Rifampicin Formulations

Same model was used for evaluation of anti-tuberculosis activity of Rifampicin in biodegradable nanoparticles. Rifampicin. in PLGA nanoparticles, given orally (twice a week, 20 mg/kg, 4 weeks treatment) was significantly more efficient in elimination of Mycobacter tuberculosis in lungs and spleen than same doses of Rifampicin solution in saline (see graph 10)

Sepsis (Septicemia) Model:

E. coli ATCC 25922 was stored at −80° C. until use in this study. The bacterium was transferred onto Trypticase Soy Agar (TSA) plates and incubated for 18 h at 37° C. A suspension of the bacterium was prepared in PBS and added to sterile 5% hog mucin. An aliquot of the suspension was added to 5% hog gastric mucin to obtain the required concentration of inoculum (3.5×10⁶ CFU/mL). Each mouse was inoculated with 0.5 mL of the appropriate inoculum preparation by IP injection. 2 hours later mice were treated with a single injection of the appropriate concentration of Gentamicin sulfate in dose 10 mg/kg (calculated by base). Animals were observed for six days and mortality was recorded.

TABLE 11
Septicemia treatment with Gentamicin in different formulations

| | E. coli | Gentamicin | | | | | |
|---|---|---|---|---|---|---|---|
| | inoculum | Dose | | Day of death | | | |
| | per mouse | (as a base) | No. dead/ | after inoculation | | | |
| Group | (actual) | mg/kg | No. treated | 1 | 2 | 3 | 4 |
| Infected & | 1.8 × 10⁶ | 0 | 8/9 | 7 | 1 | | |
| Untreated | | | | | | | |
| Control | | | | | | | |
| Gentamicin | 1.8 × 10⁶ | 10 | 5/10 | | 3 | 1 | 1 |
| sulfate | | | | | | | |
| solution | | | | | | | |
| GM in | 1.8 × 10⁶ | 10 | 1/10 | | 1 | | |
| nanoparticles | | | | | | | |
| (Example | | | | | | | |
| 42) | | | | | | | |

One of the tested formulations (Example 42, see Graph 11) showed better protection against E.Coli induced septicemia in mice than Gentamicin solution (Survival rates 90% and 50%, respectively; for untreated group survival rate is 11%)

Other Formulations:

Levofloxacin and Azithromycin in NP formulations (examples 67 and 80) showed increase levels in lungs, liver and spleen in healthy animals compared with drug solution, administrated in same doses; AUC (0-24 hr) increased 73% and 161%, respectively.

Administration of Doxorubicin in PLGA nanoparticles (example 97) in glioblastoma model improved survival rate to 40% at day 100 after tumor inoculation, while Doxorubicin in solution, administered in the same dose and schedule, did not provide any protection (0% survival).

### REFERENCES

1. Eng R H K, et al. Antibiotic killing of bacteria: comparison of bacteria on surfaces and in liquid, growing and non-growing. Chemotherapy 1995; 41:113-20
2. Huguette Pinto-Alphandary, Antoine Andremont, Patrick Couvreur International Journal of Antimicrobial Agents 13 (2000) 155-168 Targeted delivery of antibiotics using liposomes and nanoparticles: research and applications
3. U.S. Pat. No. 4,897,384 Janoff, et al. Drug preparations of reduced toxicity;
4. U.S. Pat. No. 5,759,571 Hersch, et al. Antibiotic formulation and use for drug resistant infections;
5. Schiffelers R, et al., Liposome-encapsulated aminoglycosides in pre-clinical and clinical studies. J Antimicrob Chemother. 2001 September; 48(3):333-44.
6. P. R. J. Gangadharam, et al., Chemotherapeutic potential of free and liposome encapsulated streptomycin against experimental Mycobacterium avium complex infections in beige mice Journal of Antimicrobial Chemotherapy (1991) 28, 425-435
7. Yu-Kyoung Oh, et al. Formulation and Efficacy of Liposome-Encapsulated Antibiotics for Therapy of Intracellular Mycobacterium avium Infection. Antimicrobial Agents And Chemotherapy, September. 1995, Vol. 39, No. 9 p. 2104-2111
8. Couvreur P, Roland M, Speiser P. Biodegradable submicroscopic particles containing a biologically active substance and compositions containing them. U.S. Pat. No. 4,329,332
9. K. S. Soppimath et al. Journal of Controlled Release 70 (2001) 1-20
10. E. Fattal, M. Youssef, P. Couvreur, A. Andremont Treatment of Experimental Salmonellosis in Mice with Ampicillin-Bound Nanoparticles ANTIMICROBIAL AGENTS AND CHEMOTHERAPY, p. 1540-1543 September. 1989
11. Owens D E, Peppas N A., Opsonization, biodistribution, and pharmacokinetics of polymeric nanoparticles.Int J Pharm. 2006 v. 307 no. 1 pp. 93-102.
12. U.S. Pat. No. 7,025,991 Sabel, et al. Drug targeting system, method of its preparation and its use
13. Kreuter J., et al., Direct Evidence that Polysorbate-80-Coated Poly(Butylcyanoacrylate) Nanoparticles Deliver Drugs to the CNS via Specific Mechanisms Requiring Prior Binding of Drug to the Nanoparticles; Pharmaceutical Research, Vol. 20, No. 3, pp. 409-416
14. U.S Pat. No. 6,117,454 Kreuter J., et al., “Drug targeting to the nervous system by nanoparticles”
15. Jiang-Hao Chen, Rui Ling, Qing Yao, Ling Wang, Zhong Ma, Yu Li, Zhe Wang, Hu Xu “Enhanced antitumor efficacy on hepatoma-bearing rats with adriamycin-loaded nanoparticles administered into hepatic artery” World J. Gastroenterol. 2004; v. 10 No. 13 pp. 1989-1991
16. Tracy, Mark et al., U.S. Pat. No. 7,097,857 Methods and compositions for the targeted delivery of biologically active agents
17. U.S. Pat. No. 6,749,866 Bernstein, H. et al. Modulated release from biocompatible polymers PLGA microparticles (20 mcm, 20% water soluble polymer—pore forming), polymer PLGA stabilized with cation to regulate release
18. U.S. Pat. No. 6,500,448 Johnson, O. L, et al. Composition for sustained release of human growth hormone—Zn-stabilized HGH in biodegradable polymeric matrix with CMC and Tween
19. U.S. Pat. No. 5,543,158 [19] Gref, R et al. Biodegradable injectable nanoparticles—PLGA-PEG copolymer, no additional surfactant, for antibodies and adjuvants delivery
20. U.S. Pat. No. 5,118,528 Fessi, et al. Process for the preparation of dispersible colloidal systems of a substance in the form of nanoparticles
21. US Patent Application 20030235619 Allen; Christine; et al. Polymer-lipid delivery vehicles
22. US Patent Application 20060177495Allen; Christine; et al. Polymer-lipid delivery vehicles
23. Muller R., et al U.S. Pat. No. 6,770,299. Lipid matrix-drug conjugates particle for controlled release of active ingredient
24. Penkler L, et al. U.S. Pat. No. 6,551,619 Pharmaceutical cyclosporin formulation with improved biopharmaceutical properties, improved physical quality and greater stability, and method for producing said formulation
25. Gasco M. R. U.S. Pat. No. 6,685,960 Solid lipidic nanospheres suitable to a fast internalization into cells
26. Gasco M. R. U.S. Pat. No. 6,238,694 Pharmaceutical composition in form of solid lipidic microparticles suitable to parenteral administration
27. F. Esmaeili, M. Hosseini-Nasr, M. Rad-Malekshahi, N. Samadi, F. Atyabi, R. Dinarvand, “Preparation and antibacterial activity evaluation of rifampicin-loaded poly lactide-co-glycolide nanoparticles”. Nanomedicine: Nanotechnology, Biology, and Medicine 3 (2007) pp. 161-167
28. J. Vandervoort, A. Ludwig “Biocompatible stabilizers in the preparation of PLGA nanoparticles: a factorial design study” International Journal of Pharmaceutics 238 (2002)
29. H. L. Wong, R. Bendayan, A. M. Rauth, H. Y. Xue, K. Babakhanian, X. Y. Wu “A Mechanistic Study of Enhanced Doxorubicin Uptake and Retention in Multidrug Resistant Breast Cancer Cells Using a Polymer-Lipid Hybrid Nanoparticle System” The Journal of Pharmacology and Experimental Therapeutics 2006 vol. 317, no. 3, pp. 1372-1381

### BRIEF DESCRIPTION OF THE SEVERAL VIESS OF THE DRAWINGS

Graph 1 describes increase of Streptomycin binding to nanoparticles along with increase of sucrose concentration
Graph 2 and Graph 3 demonstrate different dependence of Streptomycin release patterns from formulation type: solution, micellar solution and nanoparticulate formulations
Graph 4 presents release of Gentamicin from solution and nanoparticulate formulations
Graph 5 shows release of Rifampicin from solution and nanoparticulate formulations
Graph 6 illustrates release of Levofloxacin from solution and several nanoparticulate formulations
Graph 7 displays survival rate of mice, infected with Mycobacter Tuberculosis (H₃₇Rv, strain ATCC27294), treated with different Streptomycin formulations
Graph 8 presents results of counting number of Mycobacter tuberculosis in lungs of animals, treated with Streptomycin in nanoparticulate formulations and in solution
Graph 9 shows count of Mycobacter tuberculosis in spleen of animals, treated with Streptomycin in nanoparticulate formulations and in solution
Graph 10 reveals number count of Mycobacter tuberculosis in lungs and spleen of animals, treated with Rifampicin in nanoparticulate formulations and in solution
Graph 11 describes the survival rate in sepsis model in mice, caused with E.Coli (ATCC 25922) and treated with Gentamicin in solution and nanoparticulate formulations

1. A method for the treatment of systemic infection diseases, such as pneumonia, tuberculosis, peritonitis, endocarditis, pyelonephritis, meningitis or septicemia, caused by bacterial or protozoal infection, comprising:
a) systemic administration of an effective amount of a pharmaceutical composition comprised of biodegradable nanoparticles, said nanoparticles loaded with at least one antibacterial substance (antibiotic) or a pharmaceutically acceptable salt thereof,
b) said nanoparticles provide sustained release of incorporated antibiotic
c) said nanoparticles do not contain cyanoacrylates, the cumulative amount of administered antibiotic in the nanoparticulate formulation is several-fold lower than effective doses of the same antibiotic in conventional dosage forms

2. A method as set forth in claim 1, wherein said antibacterial substance (antibiotic) is associated with nanoparticles for 10-100%

3. A method for the treatment of systemic infection diseases, as set forth in claim 1, wherein said pharmaceutical composition is administrated by injection, infusion or other way

4. A pharmaceutical composition for the treatment of systemic infections, comprising of:
a) biodegradable nanoparticles, loaded with at least one water soluble antibiotic, wherein said nanoparticles do not contain cyanoacryla:tes
b) at least one water soluble adjuvant to increase association of the antibiotic with nanoparticles
c) at least one pharmaceutically acceptable surfactant or stabilizer

5. A pharmaceutical composition as set forth in claim 4, comprising of biodegradable nanoparticles, wherein said nanoparticles comprise of polymers and copolymers of d-lactic or l-lactic acid, glycolic acid, gamma-oxybutyric acid, caprolactone, polyesters, lipids, sterols or a combination thereof

6. A pharmaceutical composition as set forth in claim 4 wherein said surfactants and stabilizers selected from a group of pharmaceutically acceptable non-ionic surfactants and emulsifiers, anionic surfactants, polar lipids and phospholipids and does not contain polyvinyl alcohol

7. A pharmaceutical composition as set forth in claim 6 wherein said pharmaceutically acceptable non-ionic surfactants are selected from group of polyethoxylated derivatives (Polysorbates (Tween®), Brij®, Mirj®, Span®, Tocophersolan®, Cremophor®, Solutol®, LipoPEG®, Tyloxapol®, Span®, Labrasol®, Poloxamer®, Poloxamine® and similar surfactants), sugar esters, free and ethoxylated mono- and diglycerides, glycerol esters and polyglycerine esters

8. A pharmaceutical composition as set forth in claim 4, which additionally may comprise counter-ion component

9. A pharmaceutical composition as set forth in claim 8, wherein said counter-ion component selected from pharmaceutically acceptable anionic compounds, comprising cetylphosphate, dicetylphosphate, phosphatidylglycerol, phosphatidylserine, amino acids, tocopherol acid succinate, saturated, mono- and polyunsaturated fatty acids, such as capric, caproic, caprylic, lauric, palmitic, stearic, behenic, enantic, oleic, linoleic, benzoic, salicylic acid, cholesterol sulfate, cholesterol hemisuccinate, sodium cholate, cholic, deoxycholic, taurodeoxycholic, taurocholic acids, alkyl and arylsulfonates and salts thereof

10. A pharmaceutical composition as set forth in claim 4, wherein said antibacterial substance (antibiotic) is selected from a group of aminoglycosides, macrolides, rifampines, cephalosporins, fluoroquinolones, linear and cyclic antibacterial peptides

11. A pharmaceutical composition as set forth in claim 4, which additionally may comprise physiologically acceptable antioxidants

12. A pharmaceutical composition as set forth in claim 4, which additionally may comprise physiologically acceptable antibacterial preservatives

13. A pharmaceutical composition as set forth in claim 4, which additionally may comprise physiologically acceptable cryoprotectors

14. A pharmaceutical composition as set forth in claim 4, wherein said composition can be stored in a frozen state

15. A pharmaceutical composition as set forth in claim 4, wherein said composition can be stored in lyophilized state

16. A pharmaceutical composition as set forth in claim 4, wherein said water soluble adjuvant is selected from pharmaceutically acceptable water soluble ionic or non-ionic compounds

17. A water soluble ionic water soluble adjuvant as set forth in claim 16, wherein said component is selected from salts of mono- or divalent metals, such as sodium, potassium, calcium, magnesium, zinc, manganese and iron

18. A water soluble non-ionic water soluble coadjuvant as set forth in claim 16, wherein said component is selected from sugars, polyols and alcohols, such as glycerin, glucose, fructose, lactose, sucrose, trehalose, propylene glycol, polyethyleneglycols, poloxamers, polyethoxylated alcohols, polyvinylpyrrolidone, mannitol, sorbitol, isomaltol, cyclodextrins and dextrans

19. A pharmaceutical composition as set forth in claim 10, wherein said antibiotic is Streptomycin

20. A pharmaceutical composition as set forth in claim 10, wherein said antibiotic is Gentamicin

21. A pharmaceutical composition as set forth in claim 10, wherein said antibiotic is Vancomycin

22. A pharmaceutical composition as set forth in claim 10, wherein said antibiotic is Azithromycin

23. A pharmaceutical composition as set forth in claim 10, wherein said antibiotic is Clarithromycin

24. A pharmaceutical composition as set forth in claim 10, wherein said antibiotic is Rifampicin

25. A pharmaceutical composition as set forth in claim 10, wherein said antibiotic is Levofloxacin

26. A pharmaceutical composition as set forth in claim 10, wherein said antibiotic is Doxorubicin
